(19) United States
(12) Patent Application Publication
(10) Pub. No.: US 2007/0298994 A1
(43) Pub. Date: Dec. 27, 2007

(54) Use of 3-Mercapto-3-Methyl-Hexan-1-Ol for Use as Malodour Standard

(75) Inventors: Anja Finke, Holzminden (DE); Christopher Sabater-Luntzel, Holzminden (DE); Sabine Widder, Holzminden (DE); Heinz-Jurgen Bertram, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/571,494

(22) PCT Filed: Mar. 17, 2005
(86) PCT No.: PCT/EP05/52244

§ 371 Date: Aug. 29, 2007

(30) Foreign Application Priority Data
Jun. 30, 2004 (DE) 102004031589.2

(51) Int. Cl. G01N 31/00; A61K 8/00; A61L 9/00

(52) U.S. Cl. 512/1; 436/9

(57) ABSTRACT

The invention relates to the use of 3-mercapto-3-methyl-hexan-1-ol as a malodour standard. Particularly effective fragrance compositions masking sweat odour can be determined using this standard. The invention also relates to methods for checking the masking of sweat odour, methods for producing fragrance compositions masking sweat odour and personal hygiene products and air fresheners containing such compositions.

This invention relates to the field of masking sweat odour. In this connection, the invention relates to a substance particularly suitable as a malodour standard and to test methods in order to be able to check the masking of the odour of human sweat. Furthermore, the invention relates to methods for developing a fragrance product masking sweat odour, a corresponding personal hygiene product or air freshener and corresponding personal hygiene products and air fresheners themselves.

Within the framework of this invention, by masking of sweat odour is understood the complete or partial concealing of the odour of human sweat by a fragrance. A fragrance is thus within the further meaning any substance which creates an odour impression or changes the odour perception of another substance. In the narrower sense, within the framework of this invention a fragrance is a substance which creates an odour impression. A fragrance mixture within the framework of the invention is a mixture containing two or more fragrances in the narrower or broader sense, the fragrances normally being dissolved in a carrier medium. A fragrance product within the meaning of the invention is a formulated or individually produced commodity, in particular a commodity designed for consumption by end users, in which a fragrance or a fragrance mixture is incorporated in order to give the commodity a certain odour. The odour of human sweat odour and/or body odour that humans find unpleasant is described as bad odour or malodour.

Fragrance products such as perfumings and fragrance compositions have always been used to mask unpleasant odours, in particular the odour of human sweat. Developing such fragrance compositions and checking of their effectiveness in masking the odour of sweat has previously been done substantially by trial and error. Normally a sample of a fragrance or fragrance mixture which is to be checked for its ability to mask the odour of sweat (masking sample) is mixed with a sweat sample or a sample of an important fragrance or fragrance mixture for the odour of sweat (malodour standard) and the resulting odour impression determined olfactorily by a panel of assessors. The selection of a malodour standard, however, is difficult. Sweat is practically impossible to produce with constant odour intensity and quality, in constant quantity and of constant composition; it is not therefore suitable as a reproducible malodour standard. Thus by odour quality of the malodour standard is understood the type of bad odour perceived. Other fragrances used instead of sweat can again only reproduce certain odour qualities of sweat and are therefore only of limited suitability for predicting the quality of masking sweat odour by a fragrance product. Fragrance compositions composed of several fragrances can counteract this restriction, but they are often expensive to produce. Accordingly, developing a fragrance product masking sweat odour, in particular a corresponding personal hygiene product or a corresponding air freshener, is currently costly.

There is therefore a demand for a malodour standard that is as simple as possible, reproducible and characteristic of sweat odour. In addition, a method for checking the masking of sweat odour should be indicated. A further object of the invention was to indicate a method for producing a fragrance product masking sweat odour, in particular a personal hygiene product masking sweat odour or an air freshener masking e sweat odour. In addition, a fragrance product masking sweat odour itself should be indicated, in particular a personal hygiene product or an air freshener. The fragrance product sought should if possible mask the malodour without at the same time itself producing a strong odour or perfumistic impression.

The compound 3-mercapto-3-methyl-hexan-1-ol was found in natural human sweat. This compound is in fact only present in very small quantities in human sweat but produces an extremely strong odour impression and exhibits a very low threshold value. In addition, it is characteristic of the bad odour impression of body sweat. It is therefore particularly suitable for use as a malodour standard.

The odour of 3-mercapto-3-methyl-hexan-1-ol is felt to be typical of unpleasant sweat odour and body odour. This is therefore a component that is important in odour terms for human sweat yet hitherto unknown as a constituent of human sweat which plays an important role in the unpleasant sweat odour and body odour.

In higher concentrations, a very sweaty and also sulfurous, musty, unwashed note is recognised. In concentrations just above the threshold value concentration, a blackcurrant note can also be perceived.

Within the framework of this invention, therefore, by malodour is understood in particular that of 3-mercapto-3-methyl-hexan-1-ol.

By odour masking is understood the complete or partial concealing of an odour, in the present case of sweat odour and/or body odour or the odour of 3-mercapto-3-methyl-hexan-1-ol, by another fragrance or by a fragrance mixture. By odour neutralising is understood the (largely) complete masking.

In EP 1 055 667 A2, 3-mercapto-3-methyl-hexan-1-ol is mentioned as a flavouring. The taste of the racemic compound is described as passion fruit, blackcurrants and green, the (S)-enantiomer as herb-like and green and the (R)-enantiomer as grapefruit/passion fruit, blackcurrants and onion-like.

In German Offenlegungsschrift DE 2 316 456, the odour for a group of mercaptoalcohols which includes 3-mercapto-3-methyl-hexan-1-ol is generally described as pungent in high concentrations. The odour properties of 3-mercapto-3-methyl-hexan-1-ol itself, however, are not described.

The examinations carried out within the framework of the present invention showed that 3-mercapto-3-methyl-hexan-1-ol is masked by a plurality of substances greatly differing structurally from this thiol.

According to the invention, therefore, a test method for checking the masking of sweat odour is indicated by a test sample with one or more fragrances, comprising the steps:
a) producing a malodour standard mixture containing 3-mercapto-3-methyl-hexan-1-ol in an odourless carrier gas,
b) producing a test mixture containing 3-mercapto-3-methyl-hexan-1-ol and a test sample in the carrier gas, and
c) determining the overall intensity and/or the malodour impression of the malodour standard mixture in comparison to the test mixture.

The intensity of a substance or substance mixture describes the intensity of the mixture perceived by odour by trained assessors, irrespective of the quality of the odour as a bad odour or pleasant odour. The stronger a substance or substance mixture smells, the higher is the level of the intensity. The intensity is evaluated here on a scale of 1 to 9. Level 1 means odourless, 9 means very strong odour detected. The term malodour reduction value describes in the present case the difference in intensity, i.e. the difference between the bad odour (malodour) of the malodour standard mixture and the test mixture.

The test method is preferably carried out at 20° C. and 1013 mbar. Likewise it is preferred if the concentration of 3-mercapto-3-methyl-hexan-1-ol in the malodour standard mixture and the test mixture is the same. This allows a particularly good comparison of the mixtures.

The test sample according to the invention comprises preferably two or more fragrances. In this way, even complex fragrance mixtures such as for example essential oils can be checked for their suitability for masking sweat odour.

A standard solution of 3-mercapto-3-methyl-hexan-1-ol in an odourless solvent is preferably used to carry out the test method according to the invention. In this way, 3-mercapto-3-methyl-hexan-1-ol can be used to carry out the test method in an already sufficiently prediluted form of administration so that the dosing of 3-mercapto-3-methyl-hexan-1-ol is advantageously simplified. The standard solution particularly preferably contains therefore the malodour standard 3-mercapto-3-methyl-hexan-1-ol in a 0.01 to 0.5 wt. % solution, particularly preferably in a 0.1 wt. % solution, based on the total standard solution. Preferred odourless solvents are diethyl phthalate and dipropylene glycol.

In addition, it is preferred if in order to carry out the test method according to the invention the concentration of the malodour standard 3-mercapto-3-methyl-hexan-1-ol in the test and/or malodour standard mixture is at least 0.1 ng/l, preferably 0.1 to 3 ng/l and particularly preferably 0.5 to 2 ng/l. At these concentrations, the bad odour of the malodour standard is readily perceptible without however the sense of smell of an assessor being widely blocked by the malodour standard. These concentrations can be particularly readily adjusted by evaporating 5 μl of the 0.1 wt. % standard solution in 7 l air.

The malodour standard is used in particular in racemic form. In this form, 3-mercapto-3-methyl-hexan-1-ol can be readily produced and obtained. Should individual enantiomers or mixtures containing an enantiomer at a level which exceeds the level of the other enantiomers be used, the concentration of the standard solution must be adapted to the corresponding odour properties of the enantiomer used (optionally in excess).

The test method is particularly preferably performed by testing the individual test samples in containers of equal size for their suitability for concealing the odour of this 3-mercapto-3-methyl-hexan-1-ol. The individual samples are thereby each evaluated by at least 8 assessors (experts) by smelling in comparison to a sample only with 3-mercapto-3-methyl-hexan-1-ol (malodour standard mixture). This malodour standard mixture without fragrance (apart from 3-mercapto-3-methyl-hexan-1-ol) receives intensity 6. The assessors are selected on the basis of their ability to reproducibly evaluate the strengths of odours. The assessors are trained before the series of tests to recognise the odour of 3-mercapto-3-methyl-hexan-1-ol.

Likewise, according to the invention a method for developing a fragrance product masking sweat odour comprising the steps:
a) carrying out a test method according to one of the types according to the invention described previously with a number of test samples,
b) selecting such test mixtures, the intensity of which compared to the malodour standard mixture on a scale of 1 (odourless) to 9 (strong odour impression) is not more than 8 if the intensity of the malodour standard mixture is 6 and/or the malodour impression thereof in comparison to the malodour standard mixture on a scale of 1 (no malodour impression) to 9 (strong malodour impression) is not more than 3 if the malodour impression of the malodour standard mixture is 6,
   is indicated.

Those test samples which produce an increase in intensity compared to the malodour standard mixture by no more than 2 points, preferably not more than 1.5 points and particularly preferably not more than 1 point, are therefore selected.

Likewise it is preferred to select those test samples that exhibit a malodour reduction value of 3 or more, preferably of at least 3.5 and particularly preferably of at least 4.

The fragrance product according to the invention or to be used according to the invention and the test sample found according to the invention containing one or more fragrances can be combined in particularly preferred embodiments of the invention with perfume oils or incorporated in products and formulations without substantially changing the odour properties thereof.

Accordingly, also according to the invention a method for producing a personal hygiene product masking sweat odour or an air freshener masking sweat odour comprising the steps:
a) selecting a test mixture as described previously according to the invention,
b) preparing the personal hygiene product or air freshener containing the fragrance or fragrances of the selected test mixture in a concentration sufficient for masking sweat odour,
   is indicated.

In addition, according to the invention a personal hygiene product masking sweat odour or an air freshener masking sweat odour is indicated, produced in accordance with a production method according to the invention.

Particularly advantageous is a fragrance to be used according to the invention or a fragrance mixture according to the invention or to be used according to the invention with an increase in intensity less than 1.5 and malodour reduction value greater than or equal to 3.5.

Particularly preferred is a fragrance to be used according to the invention or a fragrance mixture according to the invention or to be used according to the invention with an increase in intensity less than 1 and malodour reduction value greater than or equal to 4.

A fragrance mixture according to the invention or to be used according to the invention with a content of at least 40 wt. % of fragrances to be used according to the invention based on the total fragrance mixture, is advantageous. Particularly preferred is a fragrance mixture according to the invention or to be used according to the invention which comprises a content of 50 to 100 wt. % of fragrances to be used according to the invention based on the total fragrance mixture.

For perfumistic and hedonistic reasons, it is often necessary to additionally use a small content of fragrances less effective against unpleasant sweat odour. A fragrance mixture according to the invention or to be used according to the invention with a content of fragrances to be used according to the invention of 50 to 90 wt. % is therefore preferred, particularly preferred is a content of fragrances to be used according to the invention of 50 to 80 wt. %, each based on the total fragrance mixture.

Previous perfumings and fragrance mixtures which aim to conceal sweat odour and/or body odour mostly exhibit a strong intrinsic odour and/or show an unsatisfactory odour concealment of sweat odour and/or body odour.

In EP 1 113 105 is described a mixture against human sweat odour as an additive to a perfuming which consists at least of one of the following active ingredients: beta-naphthyl methyl ether; methyl-beta-naphthyl keton; benzyl acetone; a mixture of hexahydro-4,7-methanoinden-5-yl-propionate and hexahydro-4,7-methanoinden-6-yl-propionate; gamma-methylionone; geranonitrile (3,7-dimethyl-2,6-octadiene-1-nitrile); dodecahydro-3a,6,6,9a-tetramethylnaphtho-(2,1-b)furan, ethylene brassylate, 1-cyclohexadecen-5-one and/or 1-cycloheptadecen-10-one.

Of the named compounds, the mixture of hexahydro-4,7-methanoinden-5-yl-propionate and hexahydro-4,7-methanoinden-6-yl-propionate for example was examined. This did not show any significant reduction in the bad odour based on 3-mercapto-3-methyl-hexan-1-ol, whereas geranonitrile does show a reduction in the bad odour, in fact at the same time also exhibits a strong intrinsic odour.

In JP 06-179610 are described fragrance mixtures acting against human sweat which from a group comprising at least one alcohol such as citronellol, geraniol, tetrahydrogeraniol, farnesol and/or phenylethyl dimethyl carbinol, contain at least one ester such as ethylene brassylate, coumarin, gamma-decalactone, citronellyl acetate, cis-3-hexenyl salicylate, and optionally other compounds from the class of ketones or aldehydes or the acetals or ketals thereof from the group cis-jasmone, heliotropin, musk ketone, methyl nonyl acetaldehyde. In addition, other components must be contained in the fragrance mixtures active against sweat which are selected from a plurality of individual fragrances and essential oils.

The tetrahydrogeraniol (3,7-Dimethyloctanol) tested by us leads to a malodour reduction, but at the same time also to a strong perfumistic impression, whilst the geraniol does not produce a clear reduction in the malodour.

In JP 11-286428 are described essential oils and individual fragrances for masking the body odour of older humans. Essential oils and individual fragrances were examined in regard to their masking effect in relation to a synthetic body odour mixture which consisted of dibutyl hydroxytoluene, heptanoic acid, octanoic acid, nonenal, octenal and triethyl citrate. In addition to a plurality of essential oils such as for example galbanum oil, peppermint oil, lavender oil, eucalyptus oil, ylang ylang oil, camomile oil, lime oil or rose oil, various individual fragrances such as phenyl acetaldeyhde, rose oxide, ethyl-2-methyl pentanoate, eugenol, coumarin, geranoil, citral, geranyl nitrile or cis-3-hexenyl acetate are also listed.

In JP 2003081804 is described a formulation which is suitable as a bath product and should mask body odour. Limonene, alpha-pinene, cis-3-hexen-1-ol, 1-penten-3-ol, myrcenol, octanal, nonanal, sinensal, amyl acetate, 3-phenylpropyl alcohol, citronellol, jasmal, allylcyclohexyl-oxyacetate, geranyl acetate, iso-butyl propionate, 10-undecenal, dodecanal, tripral, 1-carvone, menthone, geranyl acetone, vanillin, beta-naphthyl methyl ether and cedarwood oil are used for example for perfuming.

In JP 2003113392 is described a composition that should be used as an antiperspirant for masking body odour. Alpha-pinene, limonene, ethyl acetate, cineol, acetyl cedrene, geraniol, thymol, tetradecanal, amyl salicylate, vanillin, eucalyptus oil, jasmine oil and spearmint oil serve for example for perfuming.

In U.S. Pat. No. 5,538,719 it is described that cross-adaptation leads to structurally similar compounds being able to mutually mask odours. The masking of 3-methyl-2-hexenoic acid with a similar structure ester is described.

In WO 2002/085294 are described substances that mask the odour of low-molecular carboxylic acids occurring in the sweat. Here again, the masking or blocking action is based on cross-adaptation.

Examples of fragrances which can be the constituent of the fragrance products according to the invention or to be used according to the invention are found e.g. in S. Arctander, Perfume and Flavor Chemicals, Vols. I and II, Montclair, N.J., 1969, self-published or K. Bauer, D. Garbe and H. Surburg, Common Fragrance and Flavor Materials, 4^{th}. Ed., Wiley-VCH, Weinheim 2001.

The fragrance product according to the invention or to be used according to the invention, in particular the corresponding personal hygiene product produced according to the invention or the air freshener produced according to the invention, has a masking effect against unpleasant sweat odour of the human body, in particular against foot odour or underarm odour.

The fragrance product according to the invention or to be used according to the invention can be incorporated in all products which are suitable for masking unpleasant sweat odour; particularly advantageous are products which should remain on skin or hair (so-called leave-on products) and air fresheners.

The form of administration for leave-on products can vary greatly, e.g. as spray, gel, foam, ointment, tincture, lotion, cream, milk, oil or stick.

The following products for example can be named: deodorants (e.g. pump spray, aerosol spray containing propellant, roll-on, gel, cream, stick, foam), air fresheners (air fresheners, for example as a gel or liquid), body lotion, body cream or body mild (emulsions or microemulsions of the O/W or W/O type, or even as a multiple emulsion, e.g. O/W/O), skin oil, foot cream, hair setting lotion, hair spray, or as an alcoholic or alcoholic-aqueous solution for application to the skin.

As ingredients which can be contained in these products can be name for example:

preservatives, abrasives, anti-acne agents, skin anti-ageing agents, antibacterial agents, anticellulitis agents, anti-flaking agents, anti-inflammatory agents, anti-irritant agents, irritation-inhibiting agents, antimicrobial agents, antioxidants, astringents, sweat-inhibiting agents, antiseptic agents, antistatics, binders, buffers, carrier materials, chelating agents, cell stimulants, cleaning agents, care agents, depilatory agents, surface-active substances, surfactants (e.g. Na lauryl sulfate, ethoxylated C₁₂-C₁₈ fatty alcohols, quaternary ammonium salts), deodorising agents, antiperspirants, plasticisers, emulsifiers, enzymes, essential oils, fibres, film formers, fixatives, foaming agents, foam stabilisers, substances for preventing foaming, foam boosters, fungicides, gelatinising agents, gelling agents, hair care products, hair styling products, hair smoothing products, moisturising agents, moistening substances, moisture-retaining substances, bleaching agents, strengthening agents, stain removal agents, optical brightening agents, impregnating agents, dirt-repellant agents, friction-reducing agents, lubricants, moisture creams, ointments, opacifiers, plasticising agents, covering agents, polishes, brighteners, polymers, powders, proteins, refatting agents, grinding agents, silicones, skin soothing agent, skin cleaning agents, skin car products (e.g. bisabolol), skin-healing agents, skin-lightening agents, skin protection products, skin softening products, cooling products, skin cooling products, warming products, skin warming products, stabilisers, UV-absorbing products, UV filters, detergents, softeners, suspending agents, skin tanning products, thickening agents, vitamins (e.g. vitamin E), oils, waxes, fats, phospholipids, saturated fatty acids, single- or multiple-unsaturated fatty acids, α-hydroxyacids, polyhydroxyfatty acids, liquefiers, colorants, colour-protecting agents, pigments, anticorrosives, or other standard constituents of a cosmetic or dermatological formulation such as alcohols, polyols (e.g. glycerol), electrolytes, organic solvents or silicone derivatives.

The content of fragrance products according to the invention or to be used according to the invention based on the ready-to-use product can, depending on the type or application range of the product, be varied in wide ranges. Typically the content is in the range of 0.01 to 10 wt. %, advantageously in the range 0.05 to 5 wt. %, preferably in the range 0.1 to 2 wt. %. In air fresheners the content can also be more than 10 wt. %.

By using mixtures of the fragrances indicated, a product can be perfumed so that only a very weak scent is perceived, but at the same time the presence of an unpleasant odour of sweat or body odour is suppressed. A strong perfuming with high intensity is not necessary to achieve the desired effect.

Furthermore, there results for the remaining perfuming a gain in possibilities to perfume in any hedonistic direction without the unpleasant odour having to be further taken into account. The perfumer therefore experiences a greater level of perfume freedom.

### EXAMPLES

Unless otherwise stated, all values refer to weight.

### Example 1

1. Evidence and Threshold Value of 3-mercapto-3-methyl-hexan-1-ol

On analytical comparison of trace components of human sweat, the substance 3-mercapto-3-methyl-hexan-1-ol could be detected there by synthesis and comparison of the spectra.

The odour threshold values in air were determined for the substance (E)-3-methyl-2-hexenoic acid also occurring in sweat and 3-mercapto-3-methyl-hexan-1-ol recently found in sweat.

The odour threshold value denotes the concentration of an olfactorily perceptible substance in air at which this can be distinguished from the odourless ambient air.

Determination is carried out on a dynamic olfactometer. A sample of 20 μl each of dilute solutions of the components is placed in a container with 7 l air. The samples in the olfactometer are diluted clearly below the odour threshold by defined addition of odourless air. In the olfactometer, the concentrations in the sample air are increased little by little until all the assessors have characterised a perception. A group of 8 trained assessors determines the concentration of the samples to be distinguished in odour from air on three passes each so that the mean value of 24 measured values leads to the respective concentrations. The (E)-3-methyl-2-hexenoic acid is measured at 1% and the 3-mercapto-3-methyl-hexan-1-ol at 0.1% in odourless dipropylene glycol. The different dilutions were taken into account for calculating the threshold values. The threshold values refer to the pure substances. The threshold value of the (E)-3-methyl-2-hexenoic acid was determined at 15.1 ng/l air. The threshold value for the 3-mercapto-3-methyl-hexan-1-ol was determined at 0.015 ng/l air. All assessors evaluated the threshold value of 3-mercapto-3-methyl-hexan-1-ol lower than (E)-3-methyl-2-hexenoic acid in all passes. The threshold value is therefore significantly lower by a factor of 1000 than that of (E)-3-methyl-2-hexenoic acid.

2. Selection and Training of Assessors

A. The assessors must be able to differentiate the sweat odour from an odourless solvent (dipropylene glycol=DPG).

To do this, several cardboard smelling strips are immersed in a highly dilute solution (0.1% in DPG) of the 3-mercapto-3-methyl-hexan-1-ol. In addition, several smelling strips are immersed in DPG. Only those assessors who can perfectly differentiate the smelling strips in a test with 3-mercapto-3-methyl-hexan-1-ol—3-mercapto-3-methyl-hexan-1-ol—DPG and DPG—DPG—3-mercapto-3-methyl-hexan-1-ol take part in further tests.

B. Several concentrations of 3-mercapto-3-methyl-hexan-1-ol are then placed in a defined air space. The samples are sorted by the assessors according to intensity, i.e. odour strength. The series of concentrations must be correctly recognised and evaluated by the assessors. Assessors who have passed both tests can take part in the raw material test described in the following.

3. Raw Material Test Against Sweat Odour

The raw materials examines are either individual fragrances or essential oils. The raw materials are evaluated by the selected assessors in a predefined gaseous sample in regard to intensity and residual odour strength of the 3-mercapto-3-methyl-hexan-1-ol.

1 μl fragrance and 5 μl of the dilute 3-mercapto-3-methyl-hexan-1-ol (0.1% in DPG) respectively are placed in a container with a volume of 7 l fill with air. The samples are kept at room temperature (approx. 20° C.) for 15 h before evaluation. The individual samples are each evaluated by at least 8 assessors by smelling in comparison with a sample just with 3-mercapto-3-methyl-hexan-1-ol-solution. The mean value is then formed from the at least 8 values obtained for the respective raw material.

Refer to the definitions and explanations given above for the terms used and the evaluation scale.

As has been shown, the solvents diethyl phthalate (DEP) and dipropylene glycol (DPG) have no effect on the intensity of the bad odour or the total intensity. These solvents can therefore be used as markers for examples of applications for the remaining parts, not examined, here of a perfuming.

TABLE 1
Raw materials to be used according to the invention

| | Increase | Odour |
|---|---|---|
| | In | reduction |
| NAME | intensity | value |
| (CYCLOHEXYLOXY)-ACETIC ACID-2- | −0.5 | 4.3 |
| PROPENYL ESTER | | |
| (E ANTI)-4-(3A.4.5.6.7.7A-HEXAHYDRO-4.7- | 1.1 | 4.8 |
| METHANO-1H-INDEN-6-YLIDENE)-3- | | |
| METHYL-2-BUTANOL (>10%) + | | |
| (E SYN)-4-(3A.4.5.6.7.7A- | | |
| HEXAHYDRO-4.7-METHANO-1H-INDEN-6- | | |
| YLIDENE)-3-METHYL-2-BUTANOL (>10%) + | | |
| 4-(3A.4.5.6.7.7A-HEXAHYDRO-4.7- | | |
| METHANO-1H-INDEN-6-YL)-3-METHYL- | | |
| 3-BUTEN-2-OL (>20%) AND (E)-1- | | |
| (3A.4.5.6.7.7A-HEXAHYDRO-4.7-METHANO- | | |
| 1H-INDEN-6-YLIDENE)-3-PENTANOL (>10%) | | |
| (E)-1-(2,4,4-TRIMETHYL-2- | 1.0 | 5.0 |
| CYCLOHEXENYL)-2-BUTEN-1-ONE | | |
| (E)-11-DODECADIENYL CYANIDE | −0.9 | 3.1 |
| (E)-11-PENTADECEN-1.15-OLIDE | 0.0 | 4.0 |
| (E)-2-[1-METHYL-3-(2,6,6-TRIMETHYL-1- | 0.9 | 4.1 |
| CYCLOHEXEN-1-YL)-2-PROPENYL]-1,3- | | |
| DIOXOLANE (>70%) AND 2-[1-METHYL-3- | | |
| (2,6,6-TRIMETHYL-1-CYCLOHEXEN-1- | | |
| YL)-1-PROPENYL]-1,3-DIOXOLANE (>10%) | | |
| (E)-2-HEXENAL | −0.2 | 3.1 |
| (E)-2-METHYLBUTYRIC ACID-3,7- | 1.1 | 4.1 |
| DIMETHYL-6-OCTENYL ESTER | | |
| (E)-3-METHYL-5-PHENYL-2-PENTENE | 1.2 | 5.1 |
| NITRILE | | |
| (E)-8-CYCLOHEXADECEN-1-ONE | 0.2 | 4.0 |
| (E/Z) 5-HEXYLDIHYDRO-4-METHYL-2- | 1.2 | 4.2 |
| FURANONE | | |
| (Z)-2-METHYL-4-PROPYL-1,3-OXATHIANE | 0.0 | 3.1 |
| (Z)-2-TRIDECENE NITRILE | 1.6 | 3.8 |
| (Z)-3-(3-HEXENYLOXY)-PROPANENITRILE | 1.5 | 5.0 |
| (Z)-ALPHA.ALPHA.4-TRIMETHYL-4- | 1.0 | 3.0 |
| CYCLOHEXANE METHANOL ACETATE | | |
| (Z)-ALPHA-METHYL-4-(1-METHYLETHYL)- | 1.0 | 4.0 |
| CYCLOHEXANE METHANOL | | |
| [(3,7-DIMETHYL-6-OCTENYL)OXY]- | 1.5 | 3.8 |
| ACETALDEHYDE | | |
| 1-(2,4,-DIMETHYL-3-CYCLOHEXENYL)-2,2- | 1.4 | 3.5 |
| DIMETHYL-1-PROPANONE | | |
| 1-(CIS-2,2,6-TRIMETHYL-CYCLOHEXYL)-3- | 0.8 | 3.1 |
| PENTANOL | | |
| 1.1,2,3,3,8-HEXAMETHYL-1,2,3,5,7,8- | −2.3 | 4.3 |
| HEXAHYDRO-6-OXACYCLOPENTA[B] | | |
| NAPHTHALENE | | |
| 1.15-PENTADECANOLIDE | 1.1 | 3.6 |
| 1.1-DIETHOXY-3,7-DIMETHYL-2,6- | −0.7 | 4.3 |
| OCTADIENE | | |
| 1,3,4,5,6,7-HEXAHYDRO-7-METHOXY- | 0.5 | 4.3 |
| 1,1,5,5-TETRAMETHYL-2,4A- | | |
| METHANONAPHTHALENE | | |
| 1,3-DIMETHYL-1,4A,9,9A-TETRAHYDRO- | 1.1 | 4.1 |
| 2,4-DIOXA-FLUORENE | | |
| 1,4A,9,9A-TETRAHYDRO-2,4-DIOXA- | 1.0 | 4.3 |
| FLUORENE | | |
| 10-UNDECENAL | 1.6 | 3.3 |
| 1-HEXANOL | 1.6 | 3.6 |
| 1-ISOPROPYL-4-METHYL-BICYCLO[2.2.2] | −0.8 | 4.2 |
| OCT-5-ENE-2-CARBOXYLIC ACID METHYL | | |
| ESTER | | |
| 2,2-DIMETHYL-3-(3-METHYPHENYL)- | 1.2 | 4.4 |
| PROPANOL | | |
| 2,4,4,7-TETRAMETHYL-6-OCTEN-3-ONE | 1.0 | 3.3 |
| 2,4-DIHYDROXY-3.6-DIMETHYLBENZOIC | 1.3 | 3.8 |
| ACID METHYL ESTER | | |
| 2,4-DIHYDROXY-3-METHYLBENZOIC ACID | 0.0 | 3.0 |
| METHYL ESTER | | |
| 2,6,10-TRIMETHYL-5,9-UNDECADIENAL | 0.2 | 3.8 |
| 2,6,6-TRIMETHYL-1,3-CYCLOHEXADIENE | −2.8 | 3.0 |
| CARBOXYLIC ACID ETHYL ESTER | | |
| 2.6,9-TRIMETHYL-2,5,9- | 1.5 | 4.5 |
| CYCLODODECATRIENOL | | |
| 2-[[3-[4-1 1-DIMETHYLETHYL)PHENYL)-2- | −0.4 | 3.3 |
| METHYLPROPLIDEN]AMINO]-BENZOIC | | |
| ACID METHYL ESTER | | |
| 2-ACETYLBENZOFURAN | 1.2 | 3.3 |
| 2-ETHYL-4-(2.2.3-TRIMETHYL-3- | −1.2 | 4.5 |
| CYCLOPENTEN-1-YL-(2E)-2-BUTEN-1-OL | | |
| (<90%) AND 2-ETHYL-4-(2.2.3- | | |
| TRIMETHYL-3-CYCLOPENTEN-1-YL-(2Z)-2- | | |
| BUTEN-1-OL (<10%) | | |
| 2-HEPTYL-CYCLOPENTANONE | 1.3 | 4.5 |
| 2-METHOXY-4-METHOXYMETHYL- | −0.1 | 4.1 |
| PHENOL | | |
| 2-METHOXYMETHYLPHENOL | −2.1 | 3.8 |
| 2-METHYL-4-(2,2,3-TRIMETHYL-3- | 0.3 | 3.8> |
| CYCLOPENTENYL)-1-BUTENOL | | |
| 2-METHYL-4-(2,2,3-TRIMETHYL-3- | 0.0 | 3.0 |
| CYCLOHEXENYL)-2-BUTENOL | | |
| 2-METHYL-4-PHENYL BUTYL CYANIDE | 0.2 | 5.3 |
| 2-METHYL-5-PHENYL PENTANOL | −0.5 | 5.1 |
| 2-METHYL-PENTANOIC ACID ETHYL | 0.2 | 4.4 |
| ESTER | | |
| 2-METHYLUNDECANAL | 0.7 | 3.2 |
| 3-(4-ETHYLPHENYL)-2,2-DIMETHYL- | −1.9 | 3.7 |
| PROPIONALDEHYDE | | |
| 3-(PHENYLMETHYL)-4-HEPTANONE | 0.1 | 4.6 |
| 3,4,5,6,6-PENTAMETHYL-3-HEPTEN-2-ONE | 1.6 | 3.2 |
| 3,4-METHYLENDIOXYBENZALDEHYDE | −1.2 | 3.7 |
| 3,7-DIMETHYL-1,6-NONADIEN-3-OL | 1.0 | 3.1 |
| 3,7-DIMETHYL-6-OCTENE NITRILE | 0.5 | 4.5 |
| 3-ACETYL-3,4,10,10-TETRAMETHYL-1(6)- | 0.2 | 3.4 |
| BICYCLO[4.4.0]DECANE | | |
| 3-BUTYL-TETRAHYDRO-5-METHYL- | 1.0 | 3.0 |
| PYRAN-4-OL ACETATE | | |
| 3-ETHOXY-4-HYDROXYBENZALDEHYDE | 1.0 | 4.2 |
| 3-METHYL-4-(2,6,6-TRIMETHYL-2- | −0.7 | 5.1 |
| CYCLOHEXENYL)-3-BUTEN-2-ONE | | |
| 4-(1-METHYLETHYL)-PHENYL | 1.7 | 3.8 |
| ACETALDEHYDE | | |
| 4,7-DIHYDRO-2-(1,1.4-TRIMETHYL-3- | 0.0 | 3.7 |
| PENTENYL)-1,3-DIOXEPINE | | |
| 4,8-DIMETHYL-(3E),7-NONADIEN-2-ONE | 0.8 | 4.8 |
| (>60%) AND 4,8-DIMETHYL-(3Z),7- | | |
| NONADIEN-2-ONE (<40%) | | |
| 4.8-DIMETHYL-2-NONANONE (>40%) AND | −0.8 | 4.6 |
| 4.8-DIMETHYL-7-NONEN-2-ONE (>40%) | | |
| 4-METHOXYBENZALDEHYDE | 1.0 | 4.7 |
| 4-METHYL-4-PHENYL-2-PENTANONE | 1.4 | 4.2 |
| 4-METHYL-GAMMA-DECALACTONE | 0.9 | 5.6 |
| 5-CYCLOPENTYL-1-CYCLOPENTEN-1- | 0.3 | 4.8 |
| ACETONITRILE (<40%) AND 2- | | |
| CYCLOPENTYL-1-CYCLOPENTEN-1- | | |
| ACETONITRILE (60%) | | |
| 5-HEPTYL-2-DIHYDROFURANONE | 1.1 | 3.8 |
| 5-PENTYLDIHYDROFURAN-2-ONE | −0.8 | 4.1 |
| 6-ACETYL-1,1,2,4,4,7-HEXAMETHYL | 0.0 | 3.1 |
| TETRALINE | | |
| 8-METHYL-8-CYCLOHEXADECEN-1-ON | 0.7 | 4.5 |
| (<6 0%) AND 9-METHYL-8- | | |
| CYCLOHEXADECEN-1-ON (>40%) | | |
| ALLYL CAPRONATE | 1.0 | 3.0 |
| ALLYL HEPTYLATE | −0.5 | 3.0 |
| ALPHA.BETA.BETA.3-TETRAMETHYL- | 0.2 | 4.6 |
| BENZENE PROPANOL | | |
| ALPHA-DAMASCONE | −2.0 | 4.1 |
| ALPHA-ETHENYL-ALPHA-METHYL- | 1.0 | 4.2 |
| BENZENE PROPANE NITRILE | | |
| ALPHA-ETHYL CYCLODODECANE | 1.2 | 3.7 |
| ETHANOL (>10%) AND ALPHA.BETA- | | |
| DIMETHYL-CYCLODODECANE ETHANOL | | |
| (>80%) | | |
| ALPHA-METHYL CYCLOHEXANE | 0.8 | 4.1 |
| PROPANOL | | |
| BERGAMOTTE OIL | 1.0 | 3.0 |
| BETA.4-DIMETHYL-3-CYCLOHEXEN-1- | 0.9 | 3.9 |
| PROPANAL | | |
| BRASSYLIC ACID ETHANEDIOL ESTER | 1.1 | 3.2 |
| CIS-2,4-DIMETHYL-1-FORMYL-3- | −1.3 | 4.4 |
| CYCLOHEXENE | | |
| CROTONIC ACID-1-CYCLOHEXYL ETHYL | 0.8 | 3.7 |
| ESTER | | |
| CROTONIC ACID-2-CYCLOPENTYL | −0.8 | 4.5 |
| CYCLOPENTYL ESTER | | |
| CROTONIC ACID-4-METHYL-2- | −0.5 | 3.5 |
| PENTYLESTER | | |
| CYCLODODECANONE DIMETHYL KETAL | 1.5 | 4.0 |
| CYCLODODECYL METHYL ETHER | 0.7 | 3.1 |
| CYCLOHEXANE PROPIONIC ACID-2- | 0.1 | 4.5 |
| PROPENYL ESTER | | |
| DECANAL | 0.2 | 3.0 |
| ACETIC ACID-1-PHENYLETHYL ESTER | −0.4 | 4.0 |
| ACETIC ACID-2,4-DIMETHYL-4- | 1.6 | 3.4 |
| PHENYLPENTYL ESTER | | |
| ACETIC ACID-2-PHENYLETHYL ESTER | 1.0 | 4.0 |
| ACETIC ACID-3(E)-HEXENYL ESTER | 1.3 | 3.1 |
| ACETIC ACID-3-METHYLBUTYL ESTER | 1.3 | 5.8 |
| ACETIC ACID-ALPHA-TERPINYL ESTER | −0.3 | 4.0 |
| ACETIC-TRANS-4-TERT.-BUTYL | 0.2 | 5.1 |
| CYCLOHEXYL ESTER | | |
| EUGENOL | 0.7 | 6.0 |
| CARBONIC ACID-ETHYL-2.3.6- | 1.0 | 4.6 |
| TRIMETHYL CYCLOHEXYL ESTER | | |
| LINALOOL | 1.0 | 5.0 |
| MANDARIN OIL | 1.3 | 4.2 |
| NEROL | 1.0 | 4.0 |
| ROSEWOOD OIL | 1.0 | 5.0 |
| SALICYLIC ACID 3-METHYLBUTYL ESTER | 1.0 | 3.3 |
| SALICYLIC ACID HEXYL ESTER | 1.7 | 3.7 |
| SALICYLIC ACID PRENYL ESTER | 0.2 | 3.6 |
| TETRAHYDRO-3-PENTYL-PYRAN-4-OL | 1.5 | 4.5 |
| ACETATE | | |
| TIGLIC ACID GERANYL ESTER | 0.8 | 4.5 |
| TRANS-4-ISOPROPYL CYCLOHEXANOL | 1.0 | 4.1 |
| UNDECYL CYANIDE | 1.6 | 3.3 |
| ISOBUTYL CINNAMATE | 0.8 | 3.1 |

TABLE 2
Examples of raw materials without adequate malodour-reducing
action against 3-mercapto-3-methyl-hexan-1-ol

| | | Malodour |
|---|---|---|
| | Increase in | reduction |
| NAME | intensity | value |
| (E)-3-HEXENOL | −1.1 | 2.1 |
| (Z)-3-HEXENOL | 1.1 | 2.5 |
| 1-METHOXY-4-PROPENYL BENZENE | 2.0 | 2.0 |
| 2.4,6-TRIMETHYL-4-PHENYL-1.3- | −0.7 | 1.1 |
| DIOXANE | | |
| AMYL CINNAMALDEHYDE | 1.0 | 0.0 |
| BENZYL ACETATE | 0.4 | 2.5 |
| BENZYL ALCOHOL | 2.0 | −2.0 |
| CITRONELLOL | −0.2 | 2.2 |
| DODECANAL | 2.0 | 2.0 |
| ACETIC ACID ISOBORNYL ESTER | −0.5 | 2.3 |
| ETHYL ACETATE | −0.9 | 1.1 |
| GERANIOL | 0.7 | 2.5 |
| OCTANAL | 0.9 | 2.4 |
| PHENYL PROPYL ALCOHOL | 1.0 | 0.0 |
| PROPIONIC ACID TRICYCLO[5.2.1.0]-5(6)- | 1.0 | 2.0 |
| DECEN-2-YL ESTER | | |
| TETRAHYDRO-3-PENTYL-PYRAN-4- | 1.0 | 2.0 |
| OLACETATE | | |
| VANILLIN | 1.0 | 0.0 |
| CEDARWOOD OIL, TERPENE | 1.0 | −1.0 |
| CEDARWOOD OIL, TEXAS WHITE | 2.0 | 0.0 |

TABLE 3
Examples of raw materials with adequate malodour-reducing
action against 3-mercapto-3-methyl-hexan-1-ol, but at the same time
significant increase in intensity

| | Increase in | Malodour-reduction |
|---|---|---|
| NAME | intensity | value |
| 1,8-CINEOL | 2.4 | 5.1 |
| 3,7-DIMETHYL OCTANOL | 2.2 | 4.0 |
| AMYL SALICYLATE | 2.4 | 3.6 |
| EUCALYPTUS OIL | 3.0 | 5.0 |
| GAMMA-METHYLICONE | 2.1 | 4.0 |
| GERANYL ACETATE | 2.2 | 3.0 |
| GERANYL NITRILE | 3.0 | 5.0 |
| MENTHYL ACETATE | 2.9 | 4.4 |
| NONANAL | 2.1 | 3.0 |
| SPEARMINT OIL | 3.0 | 5.0 |
| TERPINE OL | 2.2 | 2.9 |
| TETRAHYDROLINALOOL | 2.3 | 3.0 |
| TETRAHYDROMYRCENOL | 2.2 | 4.0 |

### Example 2

### Test of Mixture/Accords Against Sweat Odour

3 mixtures (A-C) are prepared using the following recipes:

| Name | Quanity/g |
|---|---|
| Mixture A: | |
| Eugenol | 5.00 |
| Allyl capronate | 4.00 |
| 2,6,6-Trimethyl-1,3-cyclohexadiene- | 4.50 |
| carboxylic acid ethyl ester | |
| 2-Methoxy-4-methoxymethyl-phenol | 4.00 |
| 3,7-Dimethyl-6-octene nitrile | 4.50 |
| Acetic acid-trans-4-tertiär-butyl-cyclohexyl | 4.00 |
| ester | |
| (Z)-2-Methyl-4-propyl-1,3-oxathiane | 4.00 |
| DEP | 30.00 |
| Mixture B: | |
| Acetic acid-3-methylbutyl ester | 5.50 |
| 4-Methyl-gamma-decalactone | 7.00 |
| 3-Methyl-4-(2,6,6-trimethyl-2-cyclohexenyl)-3- | 4.50 |
| buten-2-one | |
| 2-Methyl-5-phenyl-pentanol | 4.50 |
| 2-Methoxymethyl phenol | 4.50 |
| 4.8-Dimethyl-2-nonanone (>40%) and 4.8- | 4.00 |
| dimethyl-7-nonen-2-one (>40%) | |
| (E)-8-Cyclohexadecen-1-one | 6.00 |
| DEP | 30.00 |
| Mixture C: | |
| 2,6,6-Trimethyl-1,3-cyclohexadiene | 7.00 |
| carboxylic acid ethyl ester | |
| 2,2-Dimethyl-3-(3-methylphenyl)-propanol | 4.50 |
| 1,4a,9.9a-Tetrahydro-2,4-dioxa-fluorene | 6.50 |
| Alpha-methylcyclohexane propanol | 5.00 |
| 2-Methoxy-4-methoxymethyl-phenol | 4.50 |
| 4,7-Dihydro-2-(1,1,4-trimethyl-3-pentenyl)- | 4.00 |
| 1,3-dioxepine | |
| 4-Methyl-4-phenyl-2-pentanone | 2.50 |
| 2-[[3-[4-(1,1-Dimethylethyl)phenyl)-2- | 6.00 |
| methylpropyliden]amino]-benzoic acid methyl | |
| ester | |
| DEP | 30.00 |

Mixtures A-C were tested in comparison to eucalyptus oil, ethyl acetate and DEP according to the above method. The mixtures have the desired action. The values for the individual substances could be repeated.

TABLE 4
Results of the tests with the mixtures

| | | Malodour reduction |
|---|---|---|
| NAME | Increase in intensity | value |
| MIXTURE A | 0.4 | 5.1 |
| MIXTURE B | −0.5 | 4.3 |
| MIXTURE C | −2.1 | 4.5 |
| EUCALYPTUS OIL | 3.2 | 5.1 |
| ETHYL ACETATE | −1.1 | 1.1 |
| DEP | −0.1 | 0.0 |

### Example 3

### Deodorant Formulation in the Form of a Roll-On Gel

| | |
|---|---|
| 1,3-Butylene glycol | 2.00 |
| PEG-40 hydrogenated castor oil | 2.00 |
| Hydroxyethyl cellulose | 0.50 |
| Fragrance mixture within the meaning of the | 0.30 |
| invention | |
| Perfume, preservatives | q.s. |
| Water | ad |
| | 100.00 |

### Example 4

### W/O Cream

| | |
|---|---|
| Paraffin oil | 10.00 |
| Ozokerite | 4.00 |
| Vaseline | 4.00 |
| Vegetable oil | 10.00 |
| Wool wax alcohol | 2.00 |
| Aluminium stearate | 0.40 |
| Fragrance mixture within the meaning of the | 1.00 |
| invention | |
| Preservatives | q.s. |
| Water | ad |
| | 100.00 |

### Example 5

### O/W Lotion

| | |
|---|---|
| Paraffin oil | 5.00 |
| Isopropyl palmitate | 5.00 |
| Cetyl alcohol | 2.00 |
| Beeswax | 2.00 |
| Ceteareth-20 | 2.00 |
| PEG-20 glyceryl stearate | 1.50 |
| Glycerol | 3.00 |
| Fragrance mixture with the meaning of the | 1.00 |
| invention | |
| Preservatives | q.s. |
| Water | ad |
| | 100.00 |

### Example 6

### Air Freshener in Gel Form

| | | |
|---|---|---|
| Demineralised water | | 89.4 |
| Genugel X-6424 (1) | Carrageenan | 2.0 |
| Fragrance mixture within the | | 0.6 |
| meaning of the invention (2) | | |
| Arkopal N 100 (3) or | Emulsifer | 3.5 |
| Tergitol NP 10 (4) | | |
| Preventol D 7 (5) | Preservative | 5.0 |

Suppliers:
(1) Hercules via: Pomosin GmbH, D-63065 Offenbach, Germany
(2) Symrise GmbH & Co. KG, 37603 Holzminden, Germany
(3) Clariant GmbH, D-60596 Frankfurt, Germany
(4) Union Carbide GmbH, D-40470 Düsseldorf, Germany
(5) Bayer AG, D-51368 Leverkusen, Germany

### Example 7

Preparation of an underarm spray for neutralising unpleasant sweat odour:

35 parts by weight dipropylene glycol, 30 parts by weight fragrance mixture according to the invention, 15 parts by weight isopropyl myristate: 15 parts by weight triethyl citrate and 10 parts by weight benzyl acetate are mixed to a composition.

A deodorant spray is prepared from 0.5 parts by weight of this composition, 40 parts by weight aqueous alcohol with 80 vol. % alcohol, 1 part by weight perfume oil (without fragrances according to the invention), 3 parts by weight isopropyl myristate and 55 parts by weight propellant (propane-butane mixture). A spray with the same composition which contains no odour-neutralising composition but 1.5 parts by weight perfume oil (without fragrances according to the invention) is prepared for comparison. On spraying the underarm area, it is shown that the spray with the fragrance mixture according to the invention reduces unpleasant sweat odour substantially better and the scent of the perfume oil is shown to better advantage.

### Example 8

### Foot Cream

| | |
|---|---|
| Soluan 5 | 2.00 |
| Methyl salicylate | 5.00 |
| Caprylic/capric triglyceride | 10.00 |
| Stearic acid | 5.00 |
| Cetyl alcohol | 1.00 |
| Glycerol | 1.00 |
| Dimethicon | 1.00 |
| Carbopol 984 | 0.50 |
| Triethanolamine | 1.50 |
| Fragrance mixture within the meaning of the | 1.00 |
| invention | |
| Perfume, preservatives | q.s. |
| Water | ad |
| | 100.00 |

### Example 9

### Hair Setting Lotion

| | |
|---|---|
| Polyvinyl pyrrolidone/vinyl acetate/ | 5.00 |
| vinyl propinale copolymer | |
| Ethanol | 45.00 |
| Fragrance mixture within the meaning of the | 1.00 |
| invention | |
| Perfume, preservatives | q.s. |
| Water | ad |
| | 100.00 |

### Example 10

### Care Mask

| | |
|---|---|
| PEG-50 lanolin | 0.50 |
| Glyceryl stearate | 2.00 |
| Sunflower seed oil | 3.00 |
| Bentonite | 8.00 |
| Kaolin | 35.00 |
| Zinc oxide | 5.00 |
| Fragrance mixture within the meaning of the | 1.00 |
| invention | |
| Preservatives | q.s. |
| Water | ad |
| | 100.00 |

### Example 11

### Skin Oil

| | |
|---|---|
| Cetyl palmitate | 3.00 |
| C₁₂₋₁₅-alkylbenzoate | 2.00 |
| Polyisobutene | 10.00 |
| Squalane | 2.00 |
| Fragrance mixture within the meaning, of the | 0.50 |
| invention | |
| Preservatives | q.s. |
| Paraffin oil | ad |
| | 100.00 |

### Example 12

### Ointment

| | |
|---|---|
| Vaseline | 36.00 |
| Ceresin | 10.00 |
| Zinc Oxide | 4.00 |
| Vegetable oil | 20.00 |
| Fragrance mixture within the meaning of the | 0.07 |
| invention | |
| Preservatives | q.s. |
| Paraffin oil | ad |
| | 100.00 |

### Example 13

### Application in the Formulations

Mixtures A-C and the fragrances of Table 4 were incorporated in the formulations of Examples 3-12 as perfume oil. The perfumed formulations were applied thinly to the front of the smelling strips and stored for three hours at room temperature. 1 μl of the 3-mercapto-3-methyl-hexan-1-ol solution (0.001% in DPG) was then added from the back and stored for a further hour at room temperature.

The coding smelling strips were evaluated sensorily by a group of assessors of at least 12 experts.

TABLE 5
Results from the application test in the formulations

| | | Increase in | Malodour |
|---|---|---|---|
| | NAME | intensity | reduction value |
| EXAMPLE 3: | MIXTURE A | 0.4 | 5.1 |
| ROLL-ON | MIXTURE B | −1.5 | 4.8 |
| DEODORANT | MIXTURE C | −2.1 | 4.5 |
| GEL | EUCALYPTUS OIL | 3.8 | 6.1 |
| | ETHYL ACETATE | −0.1 | 0.5 |
| EXAMPLE 4: | MIXTURE A | −1.0 | 3.7 |
| W/O CREAM | MIXTURE B | 0.5 | 4.1 |
| | MIXTURE C | −1.8 | 4.6 |
| | EUCALYPTUS OIL | 4.1 | 6.2 |
| | ETHYL ACETATE | 0.0 | 1.0 |
| EXAMPLE 5: | MIXTURE A | −1.0 | 3.8 |
| O/W LOTION | MIXTURE B | 0.5 | 3.5 |
| | MIXTURE C | −1.0 | 4.3 |
| | EUCALYPTUS OIL | 4.1 | 6.0 |
| | ETHYL ACETATE | 0.5 | 0.5 |
| EXAMPLE 6: | MIXTURE A | −2.1 | 4.5 |
| GEL AIR | MIXTURE B | 0.0 | 4.0 |
| FRESHENER | MIXTURE C | 1.2 | 3.0 |
| | EUCALYPTUS OIL | 4.5 | 6.5 |
| | ETHYL ACETATE | 0.5 | 0.8 |
| EXAMPLE 7: | MIXTURE A | 0.0 | 4.8 |
| AEROSOL | MIXTURE B | −1.5 | 4.8 |
| DEODORANT | MIXTURE C | −2.1 | 4.0 |
| | EUCALYPTUS OIL | 4.5 | 5.5 |
| | ETHYL ACETATE | −0.5 | 1.1 |
| EXAMPLE 8: | MIXTURE A | −1.1 | 3.7 |
| FOOT CREAM | MIXTURE B | 0.5 | 4.5 |
| | MIXTURE C | −1.5 | 4.0 |
| | EUCALYPTUS OIL | 3.1 | 4.5 |
| | ETHYL ACETATE | 0.5 | 0.8 |
| EXAMPLE 9: | MIXTURE A | 0.5 | 4.8 |
| HAIR SETTING | MIXTURE B | −1.5 | 4.8 |
| LOTION | MIXTURE C | −1.5 | 4.0 |
| | EUCALYPTUS OIL | 4.5 | 5.5 |
| | ETHYL ACETATE | −0.5 | 1.2 |
| EXAMPLE 10: | MIXTURE A | −1.0 | 3.5 |
| CARE MASK | MIXTURE B | 0.8 | 4.5 |
| | MIXTURE C | −2.5 | 3.8 |
| | EUCALYPTUS OIL | 3.6 | 5.2 |
| | ETHYL ACETATE | 0.8 | 1.2 |
| EXAMPLE 11: | MIXTURE A | −1.0 | 4.0 |
| SKIN OIL | MIXTURE B | 0.8 | 3.5 |
| | MIXTURE C | −1.5 | 4.6 |
| | EUCALYPTUS OIL | 3.3 | 5.0 |
| | ETHYL ACETATE | −0.5 | 0.3 |
| EXAMPLE 12: | MIXTURE A | −1.2 | 3.7 |
| OINTMENT | MIXTURE B | 1.3 | 4.5 |
| | MIXTURE C | −1.6 | 4.6 |
| | EUCALYPTUS OIL | 3.6 | 4.9 |
| | ETHYL ACETATE | −0.6 | 0.0 |

The mixtures according to the invention also show their action against the odour of 3-mercapto-3-methyl-hexan-1-ol in various applications but without thereby creating a strong odour impression.

1. (canceled)

2. A test method for checking the masking of sweat odour comprising the steps:
a) producing a malodour standard mixture containing 3-mercapto-3-methyl-hexan-1-ol in an odourless carrier gas,
b) producing a test mixture containing 3-mercapto-3-methyl-hexan-1-ol and a test sample in a carrier gas, and
c) determining a total intensity and/or malodour impression of the malodour standard mixture in comparison to the test mixture.

3. A method according to claim 2, wherein the malodour standard mixture contains 0.1 to 3 ng 3-mercapto-3-methyl-hexan-1-ol per litre carrier gas.

4. A method for developing a fragrance product masking sweat odour, comprising the steps:
a) carrying out the method of claim 2 with a number of test samples to form an odour scale,
b) selecting such said test mixtures, the intensity of which in comparison to the malodour standard mixture on a scale of 1 (odourless) to 9 (strong odour impression) is not more than 8 if the intensity of the malodour standard mixture is 6 and/or the malodour impression thereof in comparison to the malodour standard mixture on a scale of 1 (no malodour impression) to 9 (strong malodour impression) is not more than 3 if the malodour impression of the malodour standard mixture is 6.

5. A method for producing a personal hygiene product masking sweat odour or an air freshener masking sweat odour, comprising the steps:
a) selecting a test mixture according to claim 4 having an odour on said odour scale,
b) preparing the personal hygiene product or air freshener containing the fragrance or fragrances of the selected test mixture in a concentration sufficient for masking sweat odour.

6. A person hygiene product masking sweat odour or air freshener masking sweat odour, produced in accordance with a method according to claim 5.

7. A fragrance mixture containing at least 40 to 90 wt. % of a fragrance or a fragrance mixture which exhibits a malodour reduction value of 3 or more measured on a scale of 1 (odourless) to 9 (strong odour impression).

8. A method for making a sweat odour scale comprising the steps:
a) producing a gaseous malodour standard mixture having a predetermined concentration of 3-mercapto-3-methyl-hexan-1-ol from a solution containing 3-mercapto-3-methyl-hexan-1-ol in an odourless solvent and designating this mixture as having an intensity and malodour impression of 6 on a scale of 1 (odourless) to 9 (strong odour impression),
b) producing a first test mixture containing said malodour standard mixture and a first test sample in a carrier gas, and
c) determining a total intensity and/or malodour impression of said first test mixture relative to said malodour standard mixture.

9. A method according to claim 8 wherein said solution contains 0.01-5 wt % of 3-mercapto-3-methyl-hexan-1-ol in said odourless solvent.

10. A method according to claim 9 wherein said solution contains 0.1 wt % of 3-mercapto-3-methyl-hexan-1-ol in said odourless solvent.

11. A method according to claim 8 wherein 3-mercapto-3-methyl-hexan-1-ol is present in said gaseous malodour standard mixture in a concentration of at least 0.1 ng/l.

12. A method according to claim 8 wherein 3-mercapto-3-methyl-hexan-1-ol is present in said gaseous malodour standard mixture in a concentration within the range of 0.1 to 3 ng/l.

13. A method according to claim 8 wherein 3-mercapto-3-methyl-hexan-1-ol is present in said gaseous malodour standard mixture in a concentration within the range of 0.5 to 2 ng/l.

14. A method according to claim 8 wherein the concentration of 3-mercapto-3-methyl-hexan-1-ol in said gaseous malodour standard mixture is adjusted by evaporating 0.5 μl of said malodour standard solution in 7 litres of air.
